(19) United States
(12) Patent Application Publication
(10) Pub. No.: US 2025/0302459 A1
(43) Pub. Date: Oct. 2, 2025

(54) MEDICAL DEVICES, MEDICAL SYSTEMS, AND RELATED METHODS FOR PROVIDING TRACTION TO TISSUE

(71) Applicant: Boston Scientific Scimed, Inc., Maple Grove, MN (US)

(72) Inventors: Scott Edward CORBEIL, Litchfield, NH (US); Andrew James SCHAUBHUT, Bolton, MA (US); Austin Grant JOHNSON, Worcester, MA (US)

(73) Assignee: Boston Scientific Scimed, Inc., Maple Grove, MN (US)

(21) Appl. No.: 19/092,660

(22) Filed: Mar. 27, 2025

Related U.S. Application Data
(60) Provisional application No. 63/571,007, filed on Mar. 28, 2024.

(51) Int. Cl. A61B 17/00; A61B 1/00

(52) U.S. Cl. CPC A61B 17/00234; A61B 1/00128; A61B 1/00137; A61B 1/0014; A61B 1/00148; A61B 2017/00296; A61B 2017/00477; A61B 2017/00836; A61B 2017/00862; A61B 2017/00867

(57) ABSTRACT

Medical devices, medical systems, and related methods for providing traction to tissue are described herein. The disclosure includes a medical system. The medical system including an inner sheath defining a first lumen, an outer sheath defining a second lumen, and an anchor that is fixedly coupled to the inner sheath. The outer sheath is configured to be retracted proximally to transition the anchor from a second configuration to a first configuration. In the first configuration, the anchor is exposed and has an expanded configuration. In the second configuration, the anchor is received within the second lumen and is collapsed between the inner sheath and the outer sheath. The anchor is configured to anchor the inner sheath relative to a luminal wall.

### CROSS-REFERENCE TO RELATED APPLICATIONS

This application claims the benefit of priority to U.S. Provisional Application No. 63/571,007, filed on Mar. 28, 2024, which is incorporated by reference herein in its entirety.

### TECHNICAL FIELD

Various aspects of this disclosure generally relate to medical devices, medical systems, and related methods for providing traction to tissue. In particular, aspects of this disclosure relate to medical devices and related methods for expandable members that apply traction to tissue to facilitate resection.

### BACKGROUND

Endoscopic procedures may involve removing a lesion from a body lumen. For example, endoscopic submucosal dissection (ESD) utilizes endoscopic tools to remove cancerous or other lesions from the gastrointestinal tract. An instrument, such as an electrosurgical knife, may be passed through a working channel of an endoscope in order to cut and remove the lesion. In some procedures, a lifting agent may be injected under the lesion prior to removing the lesion, so as to facilitate access by the electrosurgical knife or other instrument. A need exists for applying traction or tension to a lesion or other portion of a body lumen to facilitate medical procedures, such as ESD.

### SUMMARY

Each of the aspects disclosed herein may include one or more of the features described in connection with any of the other disclosed aspects.

For example, the disclosure includes a medical system. The medical system may include an inner sheath. The inner sheath may define a first lumen. The medical system may include an outer sheath. The outer sheath may define a second lumen. The medical system may include an anchor. The anchor may be fixedly coupled to the inner sheath. The outer sheath may be configured to be retracted proximally to transition the anchor from a second configuration to a first configuration. In the first configuration, the anchor may be exposed and may have an expanded configuration. In the second configuration, the anchor may be received within the second lumen and may be collapsed between the inner sheath and the outer sheath. The anchor may be configured to anchor the inner sheath relative to a luminal wall.

The medical system may include one or more of the following features. The anchor may include a wire. In the first configuration, the anchor may bow radially outward relative to the inner sheath. The anchor may be a first anchor. The medical system may include a second anchor. The second anchor may include a wire. In the first configuration, the second anchor may bow radially outward relative to the inner sheath. The first anchor and the second anchor may be formed from a nickel titanium alloy.

The medical system may include one or more of the following features. A distal end of the anchor may be fixed to the inner sheath. The medical system may include a ring. The ring may be sized and shaped to receive a distal end of a scope. The medical system may include a band. The ring may be positioned at a distal end of the inner sheath. The band may be coupled the ring to the inner sheath. The ring and the band may be positioned distally to a distal end of the anchor. The ring may be configured to contact and wrap around exterior surfaces of the ring and the inner sheath.

The medical system may include one or more of the following features. The medical device may include an accessory device. The accessory device may include an end effector. The first lumen may be sized and shaped to receive the accessory device therein. The accessory device may be extendable beyond a distal end of the inner sheath.

The medical system may include one or more of the following features. The anchor may include a proximal portion. The proximal portion may extend approximately parallel to the inner sheath in the first configuration and in the second configuration.

The medical system may include one or more of the following features. The medical system may include a scope. The medical system may include an end cap at a distal end of the scope. The end cap may include a hook. The medical system may include a snare. The snare may be couplable to the hook. The first lumen of the inner sheath may be configured to receive the snare. The snare may be distally extendable from within the inner sheath to be coupled to the hook. Coupling the snare to the hook may couple the inner sheath to the end cap. In an insertion configuration of the medical system, the snare may be coupled to the hook. In the anchored configuration of the medical system, the snare may be decoupled from the hook such that an accessory device is extendible through the inner sheath. In the anchored configuration, the scope may be freely movable relative to the inner sheath.

In another example, the disclosure includes a medical system. The medical system may include an outer sheath. The outer sheath may define a first lumen. The medical system may include a shaft. The shaft may be receivable within the first lumen. The medical system may include a medical device. The medical device may include a hook, a first wing, and a second wing. The medical device may include a collapsed configuration and an expanded configuration. When in the collapsed configuration, at least a portion of each of the first wing and the second wing may be received between the outer sheath and the shaft. Retracting the outer sheath proximally relative to shaft may release the medical device from the outer sheath and the shaft, such that the medical device transitions from the collapsed configuration to the expanded configuration.

The medical system may include one or more of the following features. The medical device may include a juncture. The first wing and the second wing may be coupled to the juncture. A proximal end of the hook may be affixed to the juncture. The first wing and the second wing each may include a first leg, an inflection, and a second leg. The first leg may extend between the juncture and the inflection. The second leg may extend distally from the inflection. The hook may include a helical shape. The hook may include a sharpened distal end.

In yet another example, the disclosure includes a medical system. The medical system may include a sheath. The sheath may include a first lumen. The medical system may include a shaft. The shaft may be receivable within the first lumen. The medical system may include a medical device. The medical device may include an interlock portion and a traction portion. The traction portion may extend from a distal end of the interlock portion. The traction portion may include a first arm and a second arm. A distal end of each of the first arm and the second arm may include a barb. In a first configuration of the medical system, the medical device may be disposed within the sheath and may be coupled to a distal end of the shaft. The medical device may have a collapsed configuration, in which the first arm and the second arm may be compressed toward one another. In the second configuration, the medical device may be distal to the sheath and may be decoupled from the distal end of the shaft. The medical device may have an expanded configuration, in which the first arm and the second arm are angled away from one another. The medical device may be biased toward the expanded configuration.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts a cross sectional view of an exemplary medical system with an exemplary medical device, the medical device being in a first configuration.

FIG. 2 depicts a view of the medical device of FIG. 1 in a second configuration within a cross-section of a bodily lumen.

FIG. 3 depicts a cross sectional view of another exemplary medical system with an exemplary medical device, the medical device being in a first configuration.

FIG. 4 depicts a view of the medical device of FIG. 3 in a second configuration within cross-section of a bodily lumen.

FIG. 5 depicts another view of the medical device of FIG. 3 in the second configuration within a bodily lumen.

FIG. 6 depicts an alternative embodiment of the medical device of FIG. 3.

FIG. 7 depicts a perspective view of yet another exemplary medical system with an exemplary medical device.

FIG. 8 depicts a view of the medical device of FIG. 7 within a cross-section of a bodily lumen.

FIG. 9 depicts a perspective view of an exemplary medical system with an exemplary medical device.

FIG. 10 depicts a view of the medical system of FIG. 9 within a cross-section of a bodily lumen,

FIG. 11A depicts perspective view of the medical device of FIG. 9 in a first configuration.

FIG. 11B depicts perspective view of the medical device of FIG. 9 in a second configuration.

FIG. 12 depicts a side view of the medical system of FIG. 9 in the second configuration.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. In some of the drawings, arrows labeled “P” and “D” label proximal and distal directions, respectively.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

This disclosure is drawn to medical devices such as tissue removal devices, and related methods of use thereof.

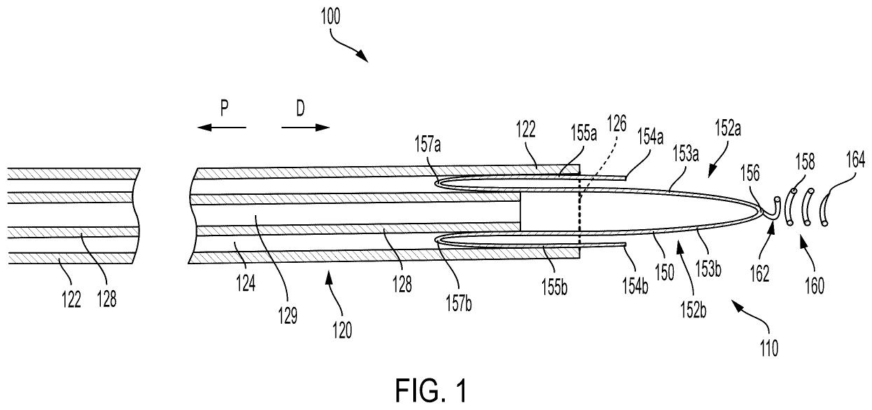

The medical devices and systems described in this disclosure may utilize shape memory components to create traction on a target and/or provide stability to a medical system, which may, in turn, apply traction to a target. In some examples, traction may be created by a shape-memory device separating and/or lifting a target (e.g., a lesion or polyp) from a surrounding area (e.g., from a surrounding wall of a bodily lumen). It may be desirable to have traction on a target to facilitate resection of the target and/or reduce the risk of an operator resecting healthy tissue or cutting through blood vessels. In other examples, a shape-memory device may provide stability to a delivery device by anchoring at least a portion of the delivery device in place relative to a bodily lumen or artificial orifice. The delivery device may be used to deliver a grasper or other traction device, which may separate and/or lift the target away from a surrounding area. It may be desirable to provide stability to the delivery device to prevent swaying, drifting, or other movement of the delivery device. The medical devices and systems shown in FIGS. 1-12 may incorporate nitinol (nickel titanium alloy) wires, nitinol wings, and/or components formed from nitinol.

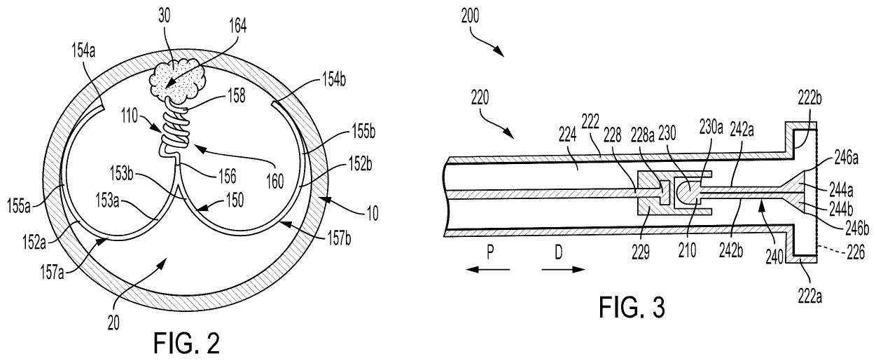

FIG. 1 depicts an exemplary medical system 100 including a medical device 110. FIG. 2 depicts medical device 110 in a bodily lumen 20. Medical system 100 may also include an insertion device 120, which may be used to deliver medical device 110 to a target site. Medical system 100 may also include a scope (such as an endoscope or an ureteroscope) (not shown). Insertion device 120 may be inserted into and extended from an opening of a distal opening of a working channel of the scope.

Insertion device 120 may include a sheath 122 (e.g., a retractable sheath). Sheath 122 may include/define a lumen 124. Sheath 122 may include a distal opening 126 positioned at a distal end of sheath 122. Distal opening 126 may be a distal opening of lumen 124. Insertion device 120 may further include a shaft 128 disposed within lumen 124. In some examples, shaft 128 may include a catheter. Shaft 128 may have a solid cross-section or may define a lumen 129, as shown in FIG. 1. Sheath 122 may be coaxial with shaft 128 and may radially surround shaft 128. Sheath 122 may be moveable relative to shaft 128. For example, sheath 122 may be retractable proximally along a longitudinal axis of shaft 128.

Medical device 110 may be received within insertion device 120. Medical device 110 may comprise biasing portion 150 and an attaching portion 160. Biasing portion 150 and attaching portion 160 may be integrally formed with one another (formed of the same piece of material) or may be formed of separate elements and fixedly coupled to one another. At least a portion of one or more of biasing portion 150 and attaching portion 160 may be formed from nitinol.

Biasing portion 150 may include one or more wings, such as a first wing 152a and a second wing 152b. First and second wings 152a, 152b may include a wire (e.g., a nitinol wire) or another shape memory element. First wing 152a and second wing 152b may be coupled/affixed together to at a juncture 156. Juncture 156 may be connected or affixed to a proximal end of biasing portion 150. First wing 152a and second wing 152b may be symmetrical to one another, as shown in FIG. 1, or may be asymmetrical. First wing 152a may include a first leg 153a and a second leg 155a. First leg 153a may extend between juncture 156 (a distal end of first leg 153a) and an inflection 157a (a proximal end of first leg 153a). Second leg 155a may extend distally from inflection 157a. Second leg 155a may include a free end 154a (an end that is not connected to another portion of biasing portion 150). Second wing 152b may include a first leg 153b and a second leg 155b. First leg 153b may extend between juncture 156 (a distal end of first leg 153b) and an inflection 157b (a proximal end of first leg 153b). Second leg 155b may extend distally from inflection 157b. Second leg 155b may include a free end 154b (an end that is not connected to another portion of biasing portion 150). Second wing 152b may include a free end 154b (an end that is not connected to another portion of biasing portion 150). Juncture 156 may be positioned between (e.g., halfway between) fee ends 154a, 154b.

Attaching portion 160 may be configured to hook, embed, pinch, tether, or otherwise attach to a target 30 (e.g., a lesion), as shown in FIG. 2. Target 30 may be attached to, embedded in, or extending from a luminal wall 10 of a bodily lumen 20. Attaching portion 160 may include a proximal end 162 and a distal end 164. Proximal end 162 may be coupled/affixed to juncture 156. Attaching portion 160 may include a hook 158. In some examples, hook 158 may be helical or spiral shaped. Hook 158 may include wire (e.g., nitinol wire) or another suitable material. Hook 158 may wind in a clockwise or counter-clockwise direction. Proximal end 162 and distal end 164 may be the proximalmost and distalmost end of hook 158, respectively. Distal end 164 may be configured to pierce through or cut through tissue. For example, distal end 164 may be sharpened. In some examples, hook 158 may include one or more barbs (not shown) along hook 158 between proximal end 162 and distal end 164, and/or one or more barbs at distal end 164. The one or more barbs may be particularly desirable in embodiments of medical device 110 where hook 158 includes a linear, straight line shape (e.g., not helical or spiral shaped) so that hook 158 may be pushed into a target without need for rotation or arcing of hook 158 while inserting. Moreover, after inserting linear, straight line hook 158, the one or more barbs may engage within the tissue of the target to prevent hook 158 from being pulled out of the target.

In some examples, hook 158 may include a multi-pronged tip (not shown). The multi-pronged tip may be positioned at distal end 164 or, alternatively, may replace hook 158 so that the multi-pronged tip extends distally from juncture 156. The multi-pronged tip may include a plurality of prongs (e.g., two or three or more prongs) and may have, for example, a forked shape. Each prong of the plurality of prongs may include one or more barbs directed between the prongs and/or directed radially inward relative to central longitudinal axis of medical device 110 and/or hook 158. Each prong of the plurality of prongs may be directed in a proximal direction or may be directed in a distal direction.

Medical device 110 may include a collapsed configuration (as seen in FIG. 1) and a deployed, expanded configuration (as seen in FIG. 2). The expanded configuration may be the default, biased, natural configuration of medical device 110. The shape-memory of first and second wings 152a, 152b may naturally transition medical device 110 from the collapsed configuration to the expanded configuration when medical device 110 is deployed from insertion device 120.

When in the collapsed configuration, medical device 110 may be at least partially received within lumen 124. As shown in FIG. 1, medical device 110 may be in a partially deployed configuration, with a portion of medical device 110 extending distally beyond distal opening 126. For example, juncture 156 and free ends 154a, 154b may extend distally beyond distal opening 126 in the partially deployed configuration. In a fully undeployed configuration, attaching portion 160 may be entirely within lumen 124. In other words, in a fully undeployed configuration, distal end 164 of attaching portion 160 may be proximal of opening 126.

In the collapsed configuration (the first, partially deployed configuration of FIG. 1 or a fully undeployed configuration), first and second wings 152a, 152b may be urged radially inward relative to a central longitudinal axis of insertion device 120 and/or medical device 110 by normal forces exerted by an interior surface of sheath 122. In other words, sheath 122 may constrain first and second wings 152a, 152b within lumen 124. At least a portion of each of first and second wings 152a, 152b may be positioned between the inner surface of sheath 122 and an exterior surface of shaft 128. In the collapsed configuration, first legs 153a, 153b may be longer than second legs 155a, 155b, such that juncture 156 is distal to free ends 154a, 154b.

When used in an exemplary medical procedure, an operator may navigate the distal end of a delivery device (e.g., an endoscopic device) to be near or adjacent to target 30 within the bodily lumen 20. The operator may extend insertion device 120 from the opening of the working channel of the endoscopic device so that the distal end of sheath 122 and/or distal end 164 of attaching portion 160 is near or adjacent to target 30. In aspects in which attaching portion 160 is fully within lumen 124 in the fully undeployed (delivery) configuration, the user may partially retract sheath 122 in order to expose attaching portion 160. The operator may attach attaching portion 160 to target 30 by piercing distal end 164 of attaching portion 160 through target 30. After distal end 164 has pierced target 30, the operator may rotate insertion device 120 (e.g., shaft 128) and/or another portion of medical device 110 in the direction that hook 158 winds (e.g., clockwise or counter-clockwise) to cause distal end 164 to pierce deeper within target 30. Distal end 164 and hook 158 may continue to be wound in a clockwise or counterclockwise direction as distal end 164 pierces deeper within target 30 via rotation of insertion device 120 (e.g., shaft 128) and/or another portion of medical device 110. Positioning distal end 164 deeper within target 30 and the clockwise or counter-clockwise winding of hook 158 within target 30 may increase a fixation of attaching portion 160 to target 30.

After medical device 110 has been attached to target 30, the operator may retract (e.g., further retract) sheath 122 in a proximal direction. As sheath 122 is retracted proximally, first wing 152a and second wing 152b may transition from the collapsed configuration to the expanded configuration of FIG. 2. Further, as sheath 122 is retracted proximally or after sheath 122 has been retracted proximally, medical device 110 may be released from insertion device 120 (e.g., sheath 122 and shaft 128). First wing 152a and second wing 152b may each include an arcuate shape while in the expanded configuration. For example, biasing portion 150 may have a rounded “W” shape in the expanded configuration. As shown in FIG. 2, in the expanded configuration, free ends 154a, 154b may extend further than juncture 156. However, such a configuration of biasing portion 150 is merely exemplary, and juncture 156 may extend further than free ends 154a, 154b.

In the expanded configuration, at least a portion of first wing 152a and second wing 152b (e.g., second legs 155a, 155b) may press against or bias against a luminal wall 10 of bodily lumen 20 (such as a wall of a colon). First wing 152a and/or second wing 152b may include surface texturing, and/or one or more protrusions (not shown) configured to help mitigate or prevent slippage or migration of medical device 110 during a medical procedure. In the expanded configuration, first and second wings 152a, 152b may be configured to generate a force on attaching portion 160 by biasing against luminal wall 10. The force on attaching portion 160 may be a force with a direction toward a center of bodily lumen 20 (away from a portion of luminal wall 10 that is adjacent to target 30). The force may be applied to target 30 by attaching portion 160, pulling target 30 in a direction toward a center of bodily lumen 20. Pulling target 30 toward the center of bodily lumen 20 (and away from surrounding areas of luminal wall 10) may generate traction that may help the operator in removing target 30 with a different medical instrument (such as hemostatic forceps, ablation devices, scissors, etc.)

FIGS. 3-5 depict an exemplary medical system 200 (FIG. 3) including a medical device 210 (FIGS. 3-5). As discussed in further detail below, medical device 210 may be or may include a clip. Medical device 210 may be deployed by using any mechanism known in the art for clips. Medical system 200 may also include an insertion device 220. Medical system 200 may also include a scope (such as an endoscope or an ureteroscopes, not shown).

Insertion device 220 may be inserted into a working channel of the scope and extended distally from a distal opening of the working channel of the scope. Medical device 210 may be received within insertion device 220 for delivery of medical device 210, as discussed below. Insertion device 220 may include a sheath 222. Sheath 222 may include/define a lumen 224. Sheath 222 may include a distal opening 226 positioned at a distal end 222a of sheath 222. Distal end 222a of sheath 222 may include a larger diameter than a remainder of sheath 222 proximal to distal end 222a. This larger diameter may, for example, accommodate an expanded configuration of medical device 210 during delivery, retrieval, and/or repositioning of medical device 210. Distal end 222a may include a shelf 222b configured to support and/or constrain a portion of medical device 210.

A shaft 228 may be disposed within lumen 224. Sheath 222 may radially surround and be approximately coaxial with shaft 228. Shaft 228 may be a wire, cable, rod, or similar structure. Shaft 228 may be moveable (e.g., proximally and distally) relative to sheath 222. As discussed below, shaft 228 may be used to deliver medical device 210.

Medical device 210 may include an interlock portion 230 and a traction portion 240. A cross section of interlock portion 230 may include a semi-circular shape or any other suitable shape. Interlock portion 230 may be interactable with a bushing 229 at a distal end 228a of shaft 228, or another device of medical system 200. Bushing 229 may have any feature of any known clip capsule or bushing known in the art. Shaft 228 and/or bushing 229 may be interactable with interlock portion 230 such that shaft 228 may be used to retrieve, reposition, re-deploy, and remove medical device 210.

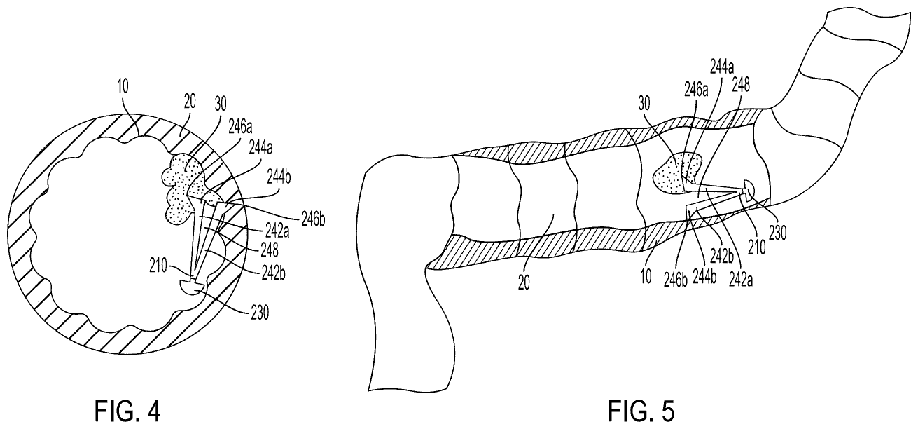

Medical device 210 may include an undeployed, collapsed configuration (as shown in FIG. 3) and a deployed, expanded configuration (as shown in FIGS. 4 and 5). A portion or an entirety of medical device 210 may be formed from a shape-memory material (e.g., nitinol). The shape-memory properties of medical device 210 may naturally transition medical device 210 from the collapsed configuration to the expanded configuration. Accordingly, the expanded configuration may be the default, natural, shape-memory configuration of medical device 210.

Traction portion 240 may extend from a distal end of interlock portion 230, for example, traction portion 240 may extend from a distal-face of interlock portion 230. Traction portion 240 may include a first arm 242a and a second arm 242b. A proximal end of each of arms 242a, 242b may be connected or affixed to the distal end of interlock portion 230. In the collapsed configuration (FIG. 3), arms 242a, 242b may be compressed or constrained toward one another, such that arms 242a, 242b may be approximately parallel to one another. In the expanded configuration (FIGS. 4-5), arms 242a, 242b may move away from one another, such that arms 242a, 242b are angled with respect to one another (in other words, arms 242a, 242b are angled away from one another).

Each of first arm 242a and second arm 242b may pivot, bend, deform, or otherwise rotate radially, relative to a central longitudinal axis of medical device 210, near interlock portion 230 and/or a distal face 230a of interlock portion 230. In the expanded configuration, traction portion 240 may include a gap 248 positioned between arms 242a, 242b. In the collapsed configuration, arms 242a, 242b (including distal ends of arms 242a, 242b) may be close to or contacting one another, such that gap 248 (not shown in FIG. 3) may be smaller or may be eliminated. Further, an angle between arms 242a, 242b may be at a minimum value in the collapsed configuration and a maximum value in the expanded configuration.

Each of arms 242a, 242b may each include a barb 244a, 244b respectively. Barbs 244a, 244b may be positioned at distal ends of arms 242a, 242b. Each of barbs 244a, 244b may be on an outer side of a respective arm 242a, 242b. In other words, barbs 244a, 244b may extend outwardly, away from a central longitudinal axis of medical device 210. Barb 244a may be disposed on arm 242a and may be on an outer side of arm 242a that faces away from arm 242b. Barb 244b may be disposed on arm 242b and may be on an outer side of arm 242b that faces away from arm 242a. Barbs 244a, 244b may each include a sharp tip 246a, 246b, respectively. Relative to the central longitudinal axis of medical device 210, sharp tips 246a, 246b may be positioned at a radially outwardmost point of barbs 244a, 244b. A diameter or dimension of each of barbs 244a, 244b may increase in the proximal-to-distal direction and a cross section of barbs 244a, 244b may resemble a triangle while medical device 210 is in the collapsed configuration (as shown in FIG. 3.) In some aspects, while medical device 210 is received within sheath 222, a portion of barbs 244a, 244b (e.g., a proximal portion of each of barbs 244a, 244b) may rest on and/or be supported by shelf 222b.

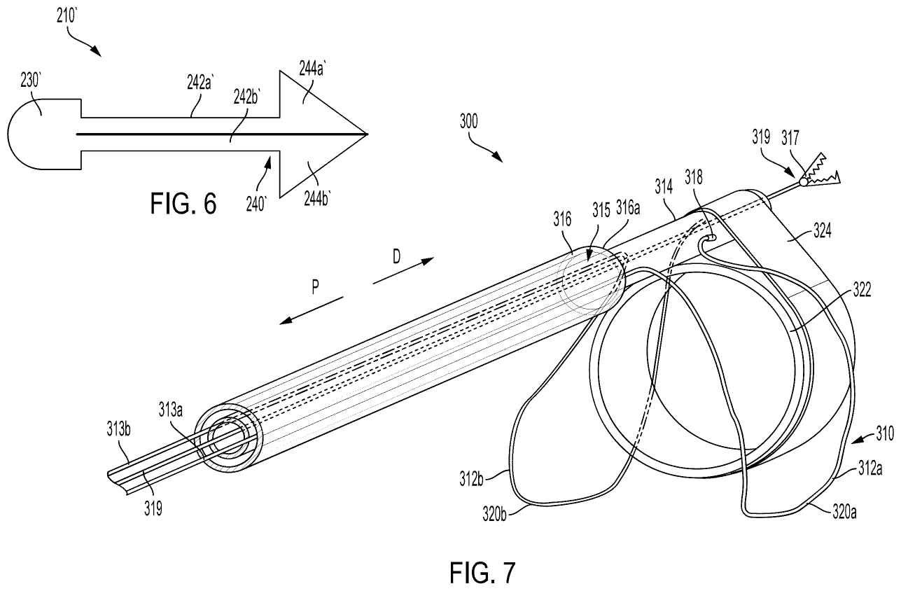

FIG. 6 depicts an alternative embodiment of medical device 210, a medical device 210′. Medical device 210′ may be used with medical system 200 in the same/similar way that medical device 210 is used with medical system 200. Medical device 210′ may include any of the features of medical device 210 and may differ as described. Medical device 210′ may include an interlock portion 230′ and a traction portion 240′. Traction portion 240′ may include a first arm 242a′ and a second arm 242b′. A proximal end of each of arms 242a′, 242b′ may be connected or affixed to the distal end of interlock portion 230′. In the collapsed configuration (FIG. 6), arms 242a′, 242b′ may be compressed or constrained toward one another, such that arms 242a′, 242b′ may be approximately parallel to one another. In the expanded configuration (similar to the expanded configuration of medical device 210′ as shown in FIGS. 4-5), arms 242a′, 242b′ may move away from one another, such that arms 242a′, 242b′ are angled with respect to one another. The diameter or dimension of each of barbs 244a′ 244b′ may decrease in the proximal-to-distal direction, beginning at a larger proximal dimension and ending in a smaller distal diameter or point. In this embodiment, arms 242a′, 242b′, including barbs 244a′, 244b′, may resemble an arrow in the collapsed configuration.

When used in an exemplary medical procedure, the operator may navigate the distal end of the scope to be near or adjacent to target 30 within bodily lumen 20. The operator may extend insertion device 220 from the opening of the working channel so that the distal end of sheath 222 and/or a distal end of traction portion 240 is near or adjacent to target 30. While medical device 210 (or medical device 210′) is received within sheath 222, medical device 210 may be in the contracted configuration due to normal forces exerted onto medical device 210 by an interior surface of sheath 222. Additionally or alternatively, bushing 229 may constrain medical device 210 in the collapsed configuration. In the collapsed configuration, gap 248 may be at a minimum dimension and/or arms 242a, 242b may be flush with one another.

The operator may deploy medical device 210 by distally extending shaft 228 so that a distal end 228a of shaft 228 urges a proximal end of interlock portion 230 in the distal direction. Prior to medical device 210 fully exiting opening 226 or after medical device 210 has fully exited opening 226, the operator may embed one of barbs 244a, 244b into luminal wall 10 of bodily lumen 20 and may embed the other of barbs 244a, 244b into a target 30 and/or a base of target 30, as shown in FIGS. 4 and 5. In some examples, as medical device 210 begins to exit through opening 226, medical device 210 may begin to transition from the collapsed configuration to the expanded configuration due to shape-memory and because sheath 222 is not constraining medical device 210. Alternatively, bushing 229 may constrain medical device 210 in the collapsed configuration, and medical device 210 may only transition from the collapsed configuration to the expanded configuration after medical device 210 is deployed from (separated from) bushing 229.

Initially, when barbs 244a, 244b are embedded in target 30 and luminal wall 10, medical device 210 may be in the collapsed configuration, or a configuration in between the collapsed configuration and the expanded configuration. As medical device 210 transitions from the collapsed configuration to the expanded configuration, arms 242a, 242b may bend radially outward relative to the central longitudinal axis of medical device 210. In other words, arms 242a, 242b may move away from one another as they transition to the expanded configuration. Due to the shape-memory properties of medical device 210, arms 242a, 242b may bias target 30 away from luminal wall 10 and generate traction on target 30. In some example, inward forces from target 30 and/or luminal wall 10 may mean that medical device 210 transitions to a partially expanded (not fully expanded) configuration before target 30 is resected, as described below.

After barbs 244a, 244b have been embedded and medical device 210 has applied traction to target 30, the operator may use a different instrument to resect target 30 from luminal wall 10. As the operator resects more of target 30 from luminal wall 10, medical device 210 may further transition to a more expanded configuration (as shown in FIG. 5). In some examples, medical device 210 may transition to a fully expanded configuration as target 30 is resected. In other words, medical device 210 may apply more traction as target 30 is resected.

An operator may use interlock portion 230 to remove or reposition medical device 210. For example, interlock portion 230 may cooperate with bushing 229 to facilitate removal and/or repositioning of medical device 210. For example, after target 30 is fully resected, medical device 210 may be removed using interlock portion 230. Alternatively, interlock portion 230 may be manipulated to reposition medical device 210 to apply traction to a different portion of target 30 or at a different angle during a procedure.

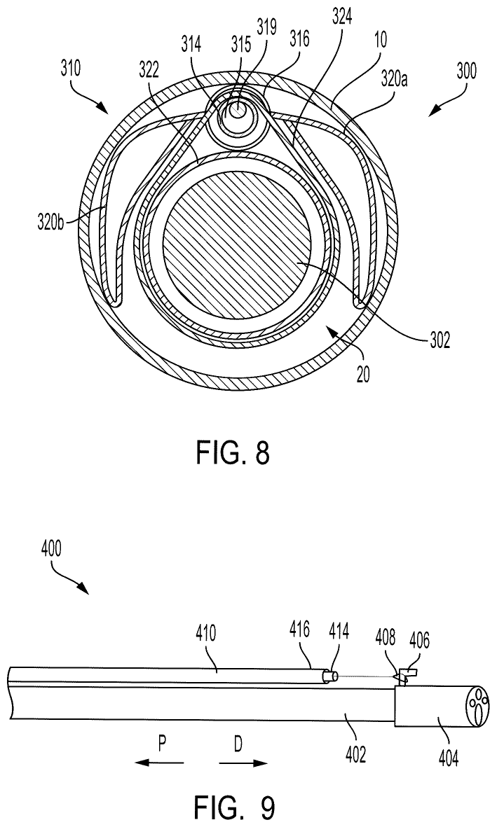

FIGS. 7-8 depict an exemplary medical system 300. Medical system 300 may include a medical device 310. Medical device 310 may be configured to receive a distal end of a scope 302 (FIG. 8) (such as an endoscope, colonoscope, ureteroscope, or any other type of endoscopic device). Medical device 310 may include one or more anchors (e.g., wires, such as nitinol wires), for example, a first anchor 312a and a second anchor 312b. When deployed, anchors 312a, 312b may contact luminal wall 10 to help stabilize medical device 310 and/or the scope within a bodily lumen.

Medical device 310 may include an inner sheath 314 and an outer sheath 316. A lumen 315 of inner sheath 314 may be configured (e.g. sized, shaped, or otherwise capable of) to removably receive an accessory device 319 having an end effector 317. At least a portion of inner sheath 314 may be received within outer sheath 316. Outer sheath 316 may be coaxial with and radially surround inner sheath 314. Outer sheath 316 may be proximally retractable relative to inner sheath 314.

Medical device 310 may further include first anchor 312a and second anchor 312b. As discussed above, in some examples, first anchor 312a and second anchor 312b may be or include shape memory wires formed of, for example, nitinol. First anchor 312a and second anchor 312b may each include a first wing portion 320a and a second wing portion 320b, respectively. First anchor 312a and second anchor 312b may further include a first proximal portion 313a and a second proximal portion 313b, respectively. In some examples, proximal portions 313a and 313b may be omitted. FIGS. 7 and 8 show anchors 312a, 312b in an expanded configuration. The expanded configuration may be the default, natural, relaxed configuration. In the expanded configuration, wing portions 320a, 320b may include a bend and/or an arcuate shape, as shown in FIGS. 7 and 8 and described in further detail below.

Wing portions 320a, 320b may be coupled to inner sheath 314 by fasteners 318 (e.g., one fastener 318 for each of wing portions 320a, 320b). Fasteners 318 may be positioned on an exterior surface of inner sheath 314 proximal to a distalmost end of inner sheath 314. Fasteners 318 may be fixedly attached to distalmost ends of anchors 312a, 312b, respectively. Wing portions 320a, 320b may be positioned adjacent to or proximal to fasteners 318. Proximal portions 313a, 313b of anchors 312a, 312b may extend along an exterior surface of inner sheath 314, approximately parallel to a longitudinal axis of inner sheath 314. In some examples, proximal portions 313a, 313b may be omitted, and proximal ends of wing portions 320a, 320b may be fixedly coupled to inner sheath 314 (e.g., via anchors having properties of fasteners 318).

Wing portions 320a, 320b may include a collapsed configuration and an expanded configuration. The shape-memory of anchors 312a, 312b may naturally transition wing portions 320a, 320b from the collapsed configuration to the expanded configuration. In the expanded configurations, anchors 312a, 312b may be exposed and wing portions 320a, 320b may bow away from inner sheath 314. Proximal and distal ends of wing portions 320a, 320b may be fixed to or adjacent to inner sheath 314, as discussed above, and portions of wing portions 320a, 320b between the proximal and distal ends may loop or bow radially outward.

Outer sheath 316 may have a first configuration, in which outer sheath 316 has a proximal position (shown in FIG. 7) and a second configuration, in which outer sheath 316 has a distal position (not shown). In the second configuration, a distal end 316a of sheath 316 may be distal to wing portions 320a, 320b and/or fasteners 318. In the second configuration, wing portions 320a, 320b may be positioned in the collapsed configuration in between an interior surface of outer sheath 316 and an exterior surface of inner sheath 314. In the second configuration, wing portions 320a, 320b may be fully or partially contained between sheath 316 and inner sheath 314.

In the first configuration, distal end 316a may be proximal to wing portions 320a, 320b. For example, distal end 316a may be proximal to an entirety of wing portions 320a, 320b (proximal to a proximalmost end of wing portions 320a, 320b). When distal end 316a is proximal to wing portions 320a, 320b, wing portions 320a, 320b may naturally transition from the collapsed configuration to the expanded configuration, shown in FIG. 7. Wing portions 320a, 320b may be transitioned to the collapsed configuration by moving sheath 316 distally to the second configuration and may be transitioned to the expanded configuration by moving sheath 316 proximally to the first configuration.

Medical device 310 may further include a saddle or ring 322. Ring 322 may be sized and shaped to receive scope 302 of medical system 300, for example a distal end of scope 302. Scope 302 may be coupled to medical device 310 once inserted into ring 322. Ring 322 may be shaped so that scope 302 can freely move proximally and distally through ring 322.

Medical device 310 may further include a band 324. Band 324 may couple ring 322 to inner sheath 314. Band 324 may contact and wrap around exterior surfaces of ring 322 and inner sheath 314 in order to couple ring 322 to inner sheath 314. Band 324 and ring 322 may be positioned distal to fasteners 318. Band 324 may be fixedly (e.g., immovably) coupled relative to ring 322 and inner sheath 314.

When used in an exemplary medical procedure, medical device 310 and scope 302 may be coupled together prior to beginning the medical procedure. The operator may navigate medical device 310 and scope 302 to a desired location within a bodily lumen of a patient (e.g., bodily lumen 20). After the operator has reached the desired location, the operator may move outer sheath 316 proximally (from the second configuration to the first configuration), allowing wing portions 320a, 320b to transition to the expanded configuration. As shown in FIG. 7, portions of wing portions 320a, 320b may contact luminal wall 10. In some examples, proximal portions 313a, 313b may be manipulated to expand and/or position wing portions 320a, 320b. Wing portions 320a, 320b may press against luminal wall 10 to provide stability to medical device 310. Providing stability to medical device 310 and scope 302 may be desirable in order to reduce unwanted swaying or movement within the bodily lumen (e.g., bodily lumen 20) and to allow the operator to introduce accessory device 319 at a desired location.

The operator may insert accessory device 319 into lumen 315 of inner sheath 314 after wing portions 320a, 320b have been expanded. Alternatively, accessory device 319 may already be disposed within lumen 315 when the operator navigates scope 302 to a treatment site. In some examples, the operator may utilize end effector 317 (which may be, for example, a forceps or a grasper) to apply tension to a target (e.g., a lesion). The operator may extend another medical device (e.g., a knife, not shown) into a working channel of scope 302 and may use the medical device to resect the target. Near the end of the procedure or when the operator no longer requires the stability provided by wing portions 320a, 320b, the operator may move outer sheath 316 to the second configuration in order to collapse wing portions 320a, 320b.

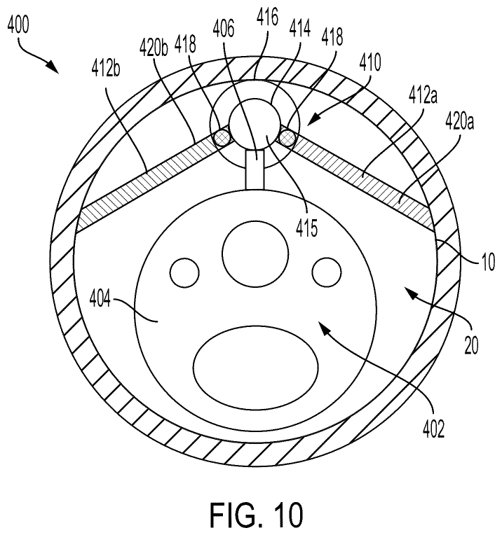

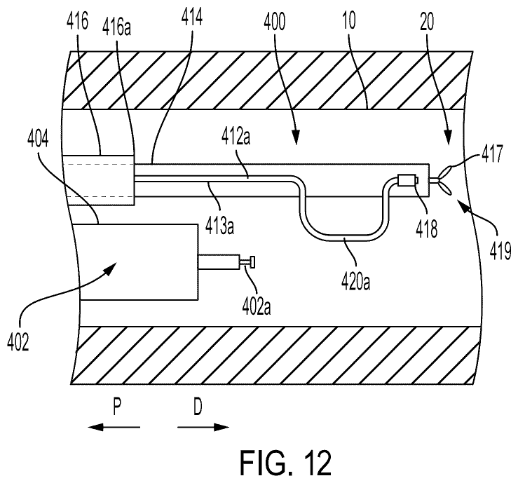

FIGS. 9-12 depict another exemplary medical system 400. Medical system 400 may include a medical device 410. Medical system 400 may also include a scope 402 (FIGS. 9, 10, and 12). An end effector 402a (e.g., a forceps, grasper, clip, or other type of end effector) may be extendable from a working channel or lumen of scope 402 (FIG. 12). Medical device 410 may be configured to be removably couplable to a distal end of scope 402 (such as an endoscope, colonoscope, ureteroscope, or any other type of endoscopic device). Medical device 410 may include one or anchors (e.g., wires, such as nitinol wires), for example, a first anchor 412a and a second anchor 412b (FIGS. 10-12). When deployed, anchors 412a, 412b may contact luminal walls 10 help stabilize medical device 410 within bodily lumen 20.

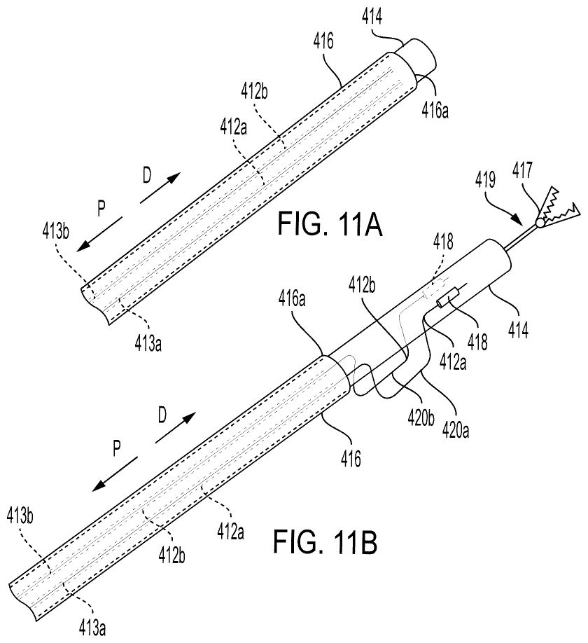

Medical device 410 may include an inner sheath 414 and an outer sheath 416. A lumen 415 of inner sheath 414 may be configured to receive an accessory device 419 having an end effector 417 (e.g., a forceps, grasper, clip, or other type of end effector), shown in FIG. 11B. At least a portion of inner sheath 414 may be received within outer sheath 416. Outer sheath 416 may be coaxial with and radially surround inner sheath 414. Outer sheath 416 may be proximally retractable relative to inner sheath 414.

Medical device 410 may further include first anchor 412a and second anchor 412b. As discussed above, in some examples, first anchor 412a and second anchor 412b may be or include shape memory wires form of, for example, nitinol. First anchor 412a and second anchor 412b may each include a first wing portion 420a and a second wing portion 420b. First anchor 412a and second anchor 412b may further include a first proximal portion 413a and a second proximal portion 413b, respectively. In some examples, proximal portions 413a and 413b may be omitted. FIGS. 9, 11B, and 12 show anchors 412a, 412b in an expanded configuration. The expanded configuration may be the default, natural, relaxed configuration. In the expanded configuration, wing portions 420a, 420b may include a bend and/or an arcuate shape, as shown in the figures and described in further detail below. Additionally, anchors 412a, 412b may include a collapsed configuration (as shown FIGS. 9 and 11A), which will be described in further detail below.

Wing portions 420a, 420b may be coupled to inner sheath 414 by fasteners 418 (e.g. one fastener 418 for each of wing portions 420a, 420b). Fasteners 418 may be positioned on an exterior surface of inner sheath 414 proximal to a distalmost end of inner sheath 414. Fasteners 418 may be fixedly attached to distalmost ends of anchors 412a, 412b, respectively. Wing portions 420a, 420b may be positioned adjacent to or proximal to fasteners 418. Proximal portions 413a, 413b of anchors 412a, 412b may extend along an exterior surface of inner sheath 414, approximately parallel to a longitudinal axis of inner sheath 414. In some examples, proximal portions 413a, 413b may be omitted, and proximal ends of wing portions 420a, 420b may be fixedly coupled to inner sheath 414 (e.g. via anchors having properties of fasteners 418).

As described above, wing portions 420a, 420b may include the collapsed configuration and the expanded configuration. The shape-memory of anchors 412a, 412b may naturally transition wing portions 420a, 420b from the collapsed configuration to the expanded configuration. In the expanded configurations, wing portions 420a, 420b may bow away from inner sheath 414. Proximal and distal ends of wings portions 420a, 420b may be fixed to or adjacent to inner sheath 414, as discussed above, and portions of wing portions 420a, 420b between the proximal and distal ends may loop or bow radially outward.

Outer sheath 416 may have a first configuration, in which outer sheath 416 has a proximal position (as shown in FIGS. 11B and 12) and a second configuration, in which outer sheath 416 has a distal position (as shown in FIGS. 9, 10, and 11A). In the second configuration, a distal end 416a of outer sheath 416 may be distal to wing portions 420a, 420b and/or fasteners 418. In the second configuration, wing portions 420a, 420b may be positioned in the collapsed configuration in between an interior surface of outer sheath 416 and an exterior surface of inner sheath 414. In the second configuration, wing portions 420a, 420b may be fully or partially contained between outer sheath 416 and inner sheath 414.

In the first configuration of outer sheath 416, distal end 416a may be proximal to wing portions 420a, 420b. For example, distal end 416a may be proximal to an entirety of wing portions 420a, 420b (proximal to a proximalmost end of wing portions 420a, 420b). When distal end 416a is proximal to wing portions 420a, 420b, wing portions 420a, 420b may naturally transition from the collapsed configuration to the expanded configuration, as shown in FIGS. 9, 11B, and 12. Wing portions 420a, 420b may be transitioned to the collapsed configuration by moving outer sheath 416 distally to the second configuration and may be transitioned to the expanded configuration by moving outer sheath 416 to the first configuration.

Scope 402 may include an end cap 404. End cap 404 may include a mating component, such as a hook 406. While the mating component is described as a hook in this disclosure, the mating component is not so limited and may be a hook, ball, cylinder, undercut, groove, or other protrusions or indentations. A snare 408, or other mating component, may be extended through inner sheath 414 during an insertion configuration of medical device 410 (e.g., before accessory device 419 is inserted into inner sheath 414). Snare 408 may be removably couplable to hook 406. When snare 408 is coupled to hook 406, inner sheath 414 and outer sheath 416 may be coupled to end cap 404 such that extending scope 402 distally may pull inner sheath 414 and outer sheath 416 in tow. In some embodiments, instead of or in additional to snare 408, end effector 417 of accessory device 419 may be removably coupled to hook 406. After end effector 417 has been coupled to hook 406, inner sheath 414 and outer sheath 416 may be coupled to end cap 404 such that extending scope 402 distally may pull inner sheath 414 and outer sheath 416 in tow. As described above, end effector 417 may include a forceps, a grasper, a clip, or other type of end effector. Utilizing end effector 417 instead of snare 408 may reduce the total number of medical devices and/or steps an operator must use and/or take in order to complete a medical procedure.

When used in an exemplary medical procedure, medical device 410 and the scope 402 may be coupled together prior to beginning the medical procedure by extending snare 408 through inner sheath 414 and coupling snare 408 to hook 406 to form an insertion configuration of the medical device. After coupling, the operator may navigate scope 402, with inner sheath 414 and outer sheath 416 in tow, to a desired location within a bodily lumen of a patient (e.g., bodily lumen 20). After the operator has reached the desired location, the operator may decouple snare 408 from hook 406 and retract snare 408 to decouple inner sheath 414 and outer sheath 416 from scope 402. In some aspects, snare 408 may be fully removed from inner sheath 414 (e.g., pulled through a proximal end of inner sheath 414). After decoupling, inner sheath 414 and outer sheath 416 may be positioned in a desirable location within bodily lumen 20. For example, as shown in FIG. 12, inner sheath 414 and outer sheath 416 may be positioned distally to a distalmost end of scope 402 so that when outer sheath 416 is moved to a first configuration, wing portions 420a, 420b would be distal to the distalmost end of scope 402. Alternatively, inner sheath 414 and outer sheath 416 may be positioned proximally to the distalmost end of scope 402, so that wing portions 420a, 420b would be proximal to the distalmost end of scope 402. After navigating inner sheath 414 and outer sheath 416 to a desirable position, the operator may move outer sheath 416 proximally (from the second configuration to the first configuration), allowing wing portions 420a, 420b to transition to the expanded configuration. Alternatively, the operator may move outer sheath 416 proximally prior to decoupling snare 407 from hook 406. When wing portions 420a, 420b are in the expanded configuration, system 400 may be in an anchored configuration. After or before wing portions 420a, 420b have transitioned to an expanded configuration, the operator may extend end effector 402a distally from scope 402 to interact with a target.

Similarly to medical system 300, portions of wing portions 420a, 420b may contact luminal wall 10. In some examples, proximal portions 413a, 413b may be manipulated (e.g., moved distally) to expand and/or position wing portions 420a, 420b. Wing portions 420a, 420b may press against luminal wall 10 to provide stability to medical device 410. Providing stability to medical device 410 may be desirable in order to reduce unwanted swaying or movement within bodily lumen 20 and to allow an operator to introduce accessory device 419 at a desired location. For example, because medical device 410 is anchored with respect to luminal wall 10, the operator may manipulate accessory device 419 while freely moving scope 402. Scope 402 may be freely movable relative to device 410, including inner sheath 414. For example, scope 402 may be retroflexed or otherwise navigated so that an operator may perform a procedure (e.g., resecting a lesion) with a device extending through scope 402.

An operator may insert accessory device 419 into lumen 415 of inner sheath 414 after wing portions 420a, 420b have been expanded. Alternatively, accessory device 419 may already be disposed within lumen 415 when the operator navigated scope 402 to a treatment site. In some examples, an operator may utilize end effector 417 (which may be, for example, a forceps or a grasper) to apply tension to a target (e.g., a lesion). The operator may extend another medical device (e.g., a knife, not shown) into a working channel of scope 402 and may use the medical device to resect the target. Near the end of the procedure or when the operator no longer requires the stability provided by wing portions 420a, 420b, the operator may move outer sheath 416 to the second configuration in order to collapse wing portions 420a, 420b.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical system comprising:
an inner sheath defining a first lumen;
an outer sheath defining a second lumen; and
an anchor that is fixedly coupled to the inner sheath;
wherein, the outer sheath is configured to be retracted proximally to transition the anchor from a second configuration to a first configuration;
wherein, in the first configuration, the anchor is exposed and has an expanded configuration;
wherein, in the second configuration, the anchor is received within the second lumen and is collapsed between the inner sheath and the outer sheath; and
wherein, the anchor is configured to anchor the inner sheath relative to a luminal wall.

2. The medical system of claim 1, wherein the anchor includes a wire, wherein, in the first configuration, the anchor bows radially outward relative to the inner sheath.

3. The medical system of claim 2, wherein the anchor is a first anchor, wherein the medical system includes a second anchor including a wire, and wherein, in the first configuration, the second anchor bows radially outward relative to the inner sheath.

4. The medical system of claim 3, wherein the first anchor and the second anchor are formed from a nickel titanium alloy.

5. The medical system of claim 1, wherein a distal end of the anchor is fixed to the inner sheath.

6. The medical system of claim 1 further comprising:
a ring sized and shaped to receive a distal end of a scope; and
a band;
wherein the ring is positioned at a distal end of the inner sheath; and
wherein the band couples the ring to the inner sheath.

7. The medical system of claim 6, wherein the ring and the band are positioned distally to a distal end of the anchor.

8. The medical system of claim 6, wherein the ring is configured to contact and wrap around an exterior surfaces of the ring and the inner sheath.

9. The medical system of claim 1 further comprising:
an accessory device including an end effector;
wherein the first lumen is sized and shaped to receive the accessory device therein; and
wherein the accessory device is extendable beyond a distal end of the inner sheath.

10. The medical system of claim 1, wherein the anchor includes a proximal portion that extends approximately parallel to the inner sheath in the first configuration and in the second configuration.

11. The medical system of claim 1 further comprising:
a scope;
an end cap at a distal end of the scope, the end cap including a hook; and
a snare;
wherein the snare is couplable to the hook.

12. The medical system of claim 11, wherein the first lumen of the inner sheath is configured to receive the snare; wherein the snare is distally extendable from within the inner sheath to be coupled to the hook.

13. The medical system of claim 12, wherein coupling the snare to the hook couples the inner sheath to the end cap.

14. The medical system of claim 11, wherein, in an insertion configuration of the medical system, the snare is coupled to the hook and wherein, in an anchored configuration of the medical system, the snare is decoupled from the hook such that an accessory device is extendible through the inner sheath.

15. The medical system of claim 14, wherein, in the anchored configuration, the scope is freely movable relative to the inner sheath.

16. A medical system comprising:
an outer sheath defining a first lumen;
a shaft receivable within the first lumen; and
a medical device comprising:
a hook,
a first wing, and
a second wing;
wherein, the medical device includes a collapsed configuration and an expanded configuration;
wherein, when in the collapsed configuration, at least a portion of each of the first wing and the second wing is received between the outer sheath and the shaft; and
wherein, retracting the outer sheath proximally relative to shaft releases the medical device from the outer sheath and the shaft, such that the medical device transitions from the collapsed configuration to the expanded configuration.

17. The medical system of claim 16, wherein the medical device includes a juncture, wherein the first wing and the second wing are coupled to the juncture, and wherein a proximal end of the hook is affixed to the juncture.

18. The medical system of claim 17, wherein the first wing and the second wing each include a first leg, an inflection, and a second leg, wherein the first leg extends between the juncture and the inflection, wherein the second leg extends distally from the inflection.

19. The medical system of claim 18, wherein, the hook includes a helical shape, and wherein the hook includes a sharpened distal end.

20. A medical system comprising:
a sheath defining a first lumen;
a shaft receivable within the first lumen; and
a medical device comprising:
an interlock portion, and
a traction portion, the traction portion extending from a distal end of the interlock portion, wherein the traction portion includes a first arm and a second arm, and wherein a distal end of each of the first arm and the second arm includes a barb;
wherein, in a first configuration of the medical system, the medical device is disposed within the sheath and coupled to a distal end of the shaft, and the medical device has a collapsed configuration, in which the first arm and the second arm are compressed toward one another;
wherein, in a second configuration, the medical device is distal to the sheath and decoupled from the distal end of the shaft, and the medical device has an expanded configuration, in which the first arm and the second arm are angled away from one another; and
wherein, the medical device is biased toward the expanded configuration.
